(19) United States
(12) Patent Application Publication
(10) Pub. No.: US 2004/0192652 A1
(43) Pub. Date: Sep. 30, 2004

(54) Pharmaceutical combinations and methods for the treatment of leukemia

(76) Inventors: Francis J. Giles, Houston, TX (US); Srdan Verstovsek, Houston, TX (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/729,387

(22) Filed: Dec. 8, 2003

Related U.S. Application Data
(60) Provisional application No. 60/431,196, filed on Dec. 6, 2002.

(51) Int. Cl. A61K 31/675; A61K 31/513

(52) U.S. Cl. 514/81; 514/269; 514/85

(57) ABSTRACT

In accordance with the present invention there is provided a pharmaceutical combination useful for the treatment of leukemia comprising at least one active compound of formula (I):

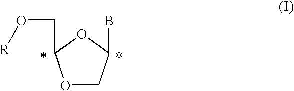

and a Bcr-Abl tyrosine kinase inhibitor, and a method of treating a patient having leukemia comprising at least one active compound of formula (I), as defined above, and a Bcr-Abl tyrosine kinase inhibitor.

This application claims the benefit of U.S. Provisional Application No. 60/431,196, filed Dec. 6, 2002, which is expressly incorporated by reference herein.

### FIELD OF THE INVENTION

The present invention relates to pharmaceutical combinations and methods useful in the treatment of leukemia. Particularly, the combinations of this invention relate to dioxolane nucleoside analogues with a Bcr-Abl tyrosine kinase inhibitor.

### BACKGROUND OF THE INVENTION

Leukemia is a malignant cancer of the bone marrow and blood. It is characterized by the uncontrolled growth of blood-cells. The common types of leukemia are divided into four categories: acute or chronic myelogenous, involving the myeloid elements of the bone marrow (white cells, red cells, megakaryocytes) and acute or chronic lymphocytic, involving the cells of the lymphoid lineage.

Standard treatment for leukemia usually involves chemotherapy and/or bone marrow transplantation and/or radiation therapy. Treatment of leukemia is very complex and depends upon the type of leukemia. Tremendous clinical variability among remissions is also observed in leukemic patients, even those that occur after one course of therapy. Patients who are resistant to therapy have very short survival times, regardless of when the resistance occurs. Despite improvements in outcome with current treatment programs, the need to discover novel agents for the treatment of all types of leukemia continues.

The two major types of bone marrow transplants are autologus (uses the patient's own marrow) and allogeneic (uses marrow from a compatible donor). Radiation therapy, which involves the use of high-energy rays, is usually given before bone marrow transplantation to kill all leukemic cells.

In treament by chemotherapy, depending on the type of leukemia, patients may receive a single drug or a combination of two or more drugs. Approximately 40 different drugs are now being used in the treatment of leukemia either alone or in combination. Some common combinations include cytarabine with either doxorubicin or daunorubicin or mitoxantrone or thioguanine, mercaptopurine with methotrexate, mitroxantrone with etoposide, asparaginase with vincristine, daunorubicin and prednisone, cyclophosphamide with vincristine, cytarabine and prednisone, cyclophosphamide with vincristine and prednisone, daunorubicin with cytarabine and thioguanine and daunorubicin with vincristine and prednisone.

Nucleoside analogues, such as cytarabine, fludarabine, gemcitabine and fludarabine represent a class of drugs having an important role in the treatment of leukemia. β-L-OddC ((−)-β-L-Dioxolane-Cytidine, Troxatyl™, troxacitabine) from Shire BioChem Inc. is also a nucleoside analogue which has been shown to have potent antitumor activity (K. L. Grove et al., Cancer Res., 55(14), 3008-11, 1995; K. L. Grove et al., Cancer Res., 56(18), 4187-4191, 1996, K. L. Grove et al., Nucleosides Nucleotides, 25 16:1229-33, 1997; S. A Kadhim et al., Can. Cancer Res., 57(21), 4803-10, 1997). In clinical studies, β-L-OddC has been reported to have significant activity in patients with advanced leukemia (Giles et al., J. Clin. Oncology, Vol 19, No 3, 2001).

More recently, STI-571 (Gleevec™, imatinib mesylate, from Novartis Pharmaceuticals Corp.) a Bcr-Abl tyrosine kinase inhibitor has shown significant antileukemic activity and specifically in chronic myeologenous leukemia. STI-571 has become a promising therapy in the group of patients targeting Bcr-Abl tyrosine kinase inhibition. However, despite significant hematologic and cytogenic responses, resistance occurs particularly in the advanced phases of chronic myelogenous leukemia.

In recent studies, combinations of STI-571 and cytarabine and homoharringtonine (HHT) have been evaluated in their in vitro effects on the activity in CML. Cancer, May 15, 2002, Volume 94, Number 10, pp 2653-2662. Recent reports have also similarly confirmed the favorable interaction between STI-571 and cytarabine.

Despite improvements in the outcome of patients with current combination treatment programs and the promising results of in vitro combinations evaluated to date, there exists a need to find other combinations of drugs which exhibit potent activity in leukemia and also which can be used in the treatment of leukemia where resistance to the present therapy has occurred.

The present invention provides a combination therapy using β-L-OddC and a Bcr-Abl tyrosine kinase inhibitor useful for the treatment of leukemia and also in the treatment of resistant-leukemia.

### SUMMARY OF THE INVENTION

In one aspect, the present invention provides a novel pharmaceutical combination useful for the treatment of leukemia comprising at least one active compound of formula (1):

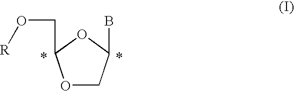

or a pharmaceutically acceptable salt thereof,

wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and

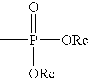

wherein each Rc is independently selected from the group comprising H, C₁₋₆ alkyl, C₂₋₆ alkenyl, C₂₋₆ alkynyl and a hydroxy protecting group;

and a Bcr-Abl tyrosine kinase inhibitor.

In one aspect, the present invention provides a novel pharmaceutical combination useful for the treatment of leukemia comprising at least one active compound of formula (1):

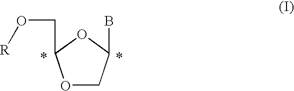

or a pharmaceutically acceptable salt thereof,

wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and

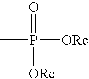

wherein each Rc is independently selected from the group comprising H, C₁₋₆ alkyl, C₂₋₆ alkenyl, C₂₋₆ alkynyl and a hydroxy protecting group;

and imatinib mesylate.

The pharmaceutical combinations of the present invention are useful in the treatment of leukemia, in particular in the treatment of leukemia selected from the group comprising acute myelogenous leukemia (AML), chronic myelogenous leukemia (CML), acute lymphocytic leukemia (ALL), chronic lymphocytic leukemia (CLL) and hairy cell leukemia (HCL).

In another aspect, the pharmaceutical combinations of the present invention are useful in the treatment of leukemia, in particular in the treatment of CML.

In another aspect, the pharmaceutical combinations of the present invention are useful in the treatment of leukemia, in particular in the treatment of CML which is resistant to current drug therapy.

In another aspect, there is provided a method of treating a patient having leukemia comprising administering to said patient a therapeutically effective amount of a compound of formula (I) in combination with a Bcr-Abl tyrosine kinase inhibitor and at least one further therapeutic agent.

In another aspect, there is provided a method of treating a patient having cancer, in particular in the treatment of refractory leukemia comprising administering to said patient having refractory leukemia a therapeutically effective amount of a compound of formula (I) and at least one further therapeutic agent. Preferably, the further therapeutic agent is other than doxorubicin. Also, the ratio of the compound of formula (I) to the further therapeutic agent is preferably 1:250 to 250:1, more preferably 1:50 to 50:1, especially 1:20 to 20:1.

In another aspect, there is provided a pharmaceutical formulation comprising the combination of the compound of formula (I) and at least one further therapeutic agent in combination with at least a pharmaceutically acceptable carrier or excipient. Preferably, the further therapeutic agent is other than doxorubicin. Also, the ratio of the compound of formula (I) to the further therapeutic agent is preferably 1:250 to 250:1, more preferably 1:50 to 50:1, especially 1:20 to 20:1.

Another aspect of the invention is the use of a compound according to formula (I) and at least one further therapeutic agent, for the manufacture of a medicament for treating cancer in a mammal. Preferably, the further therapeutic agent is other than doxorubicin. Also, the ratio of the compound of formula (I) to the further therapeutic agent is preferably 1:250 to 250:1, more preferably 1:50 to 50:1, especially 1:20 to 20:1.

### DESCRIPTION OF THE FIGURES

FIG. 1 represents the graphical representation of the MTS assay evaluating the combination of β-L-OddC and STI-571 (imatinib mesylate) in the KBM-5 cell line.

FIG. 2 represents the graphical representation of the MTS assay evaluating the combination of β-L-OddC and STI-571 (imatinib mesylate) in the KBM5-STI resistant cell line.

FIG. 3 represents the graphical representation of the MTS assay evaluating the combination of β-L-OddC and STI-571 (imatinib mesylate) in the KBM-7 cell line.

FIG. 4 represents the graphical representation of the MTS assay evaluating the combination of β-L-OddC and STI-571 (imatinib mesylate)in the KBM-7-STI resistant cell line.

FIGS. 5 and 6 represent the graphical results of the evaluation in the Caspase 3/7 assay of the combination of β-L-OddC and STI-571 (imatinib mesylate) in the KBM5-STI resistant cell line at 48 hrs and 72 hrs, respectively.

FIGS. 7 to 11 represent the graphical results of the evaluation in the Caspase 3/7 assay of the combination of β-L-OddC and STI-571 (imatinib mesylate) in the KBM7-STI resistant cell line at 6 hrs, 10 hrs, 27 hrs, 48 hrs and 72 hrs, respectively.

FIGS. 12, 13 and 14 represent the results of the comparative in vivo antitumor activity of β-L-OddC with or without STI-571 (imatinib mesylate) treatment in mice bearing KBM-5 or KBM-5-STI resistant chronic myeloid leukemia cells.

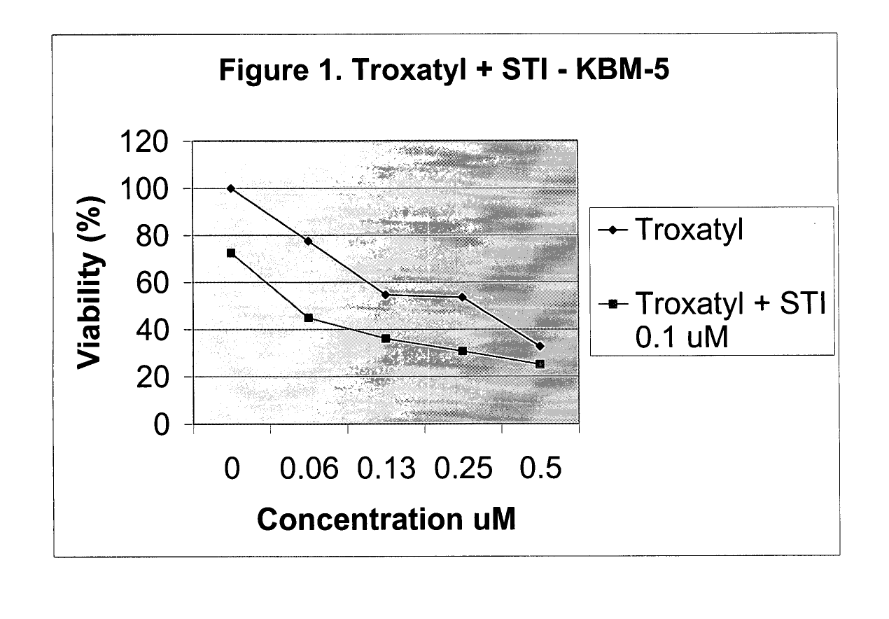

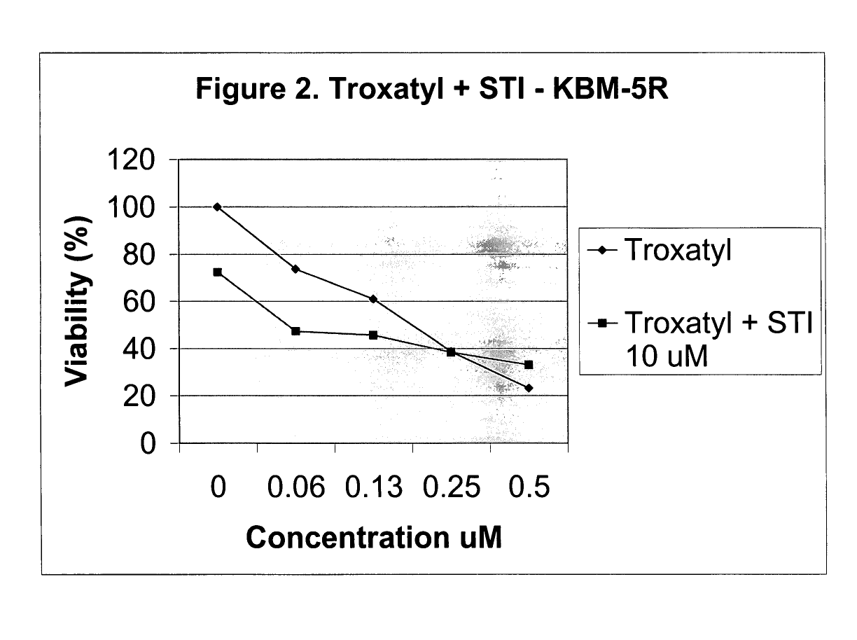

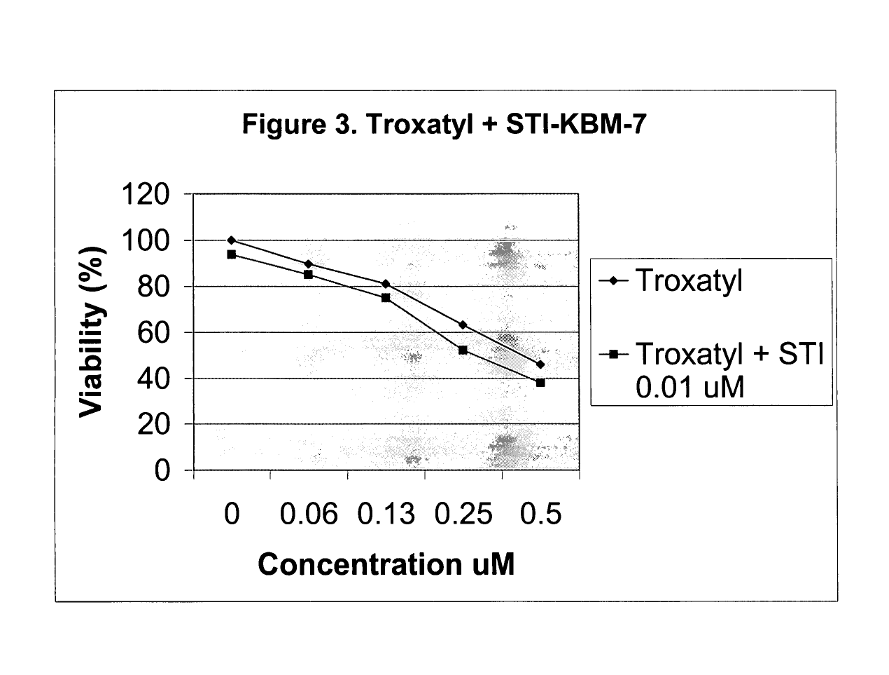

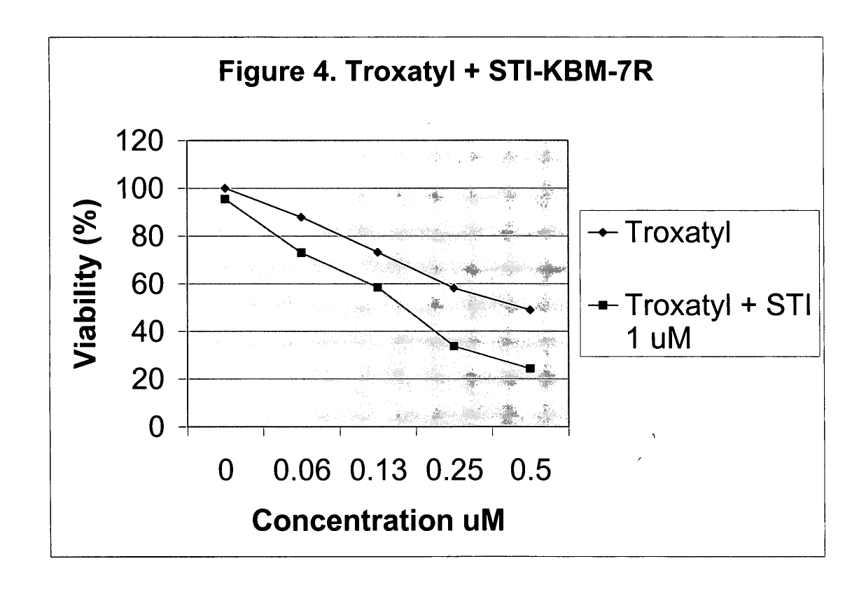

### DETAILED DESCRIPTION OF THE INVENTION

The present invention provides a novel pharmaceutical combination useful for the treatment of leukemia in a mammal comprising at least one active compound of formula (I):

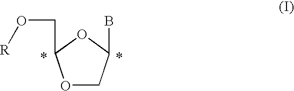

or a pharmaceutically acceptable salt thereof, wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and

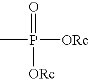

wherein each Rc is independently selected from the group comprising H, C₁₋₆ alkyl, C₂₋₆ alkenyl, C₂₋₆ alkynyl and a hydroxy protecting group;

and a Bcr-Abl tyrosine kinase inhibitor.

In one embodiment, R is H.

In one embodiment, B is cytosine.

In one embodiment, R is H and B is cytosine.

In one embodiment, B is 5-fluorocytosine.

In one embodiment, a compound of formula I is (−)-β-L-Dioxolane-Cytidine (β-L-OddC).

In one embodiment, a compound of formula I is (−)-β-Dioxolane-5-fluoro-Cytidine (5-FddC).

In another embodiment, the compounds of formula (I) of the present invention is substantially in the form of the (−) enantiomer.

In a further embodiment, the compounds formula (I) present in the pharmaceutical combination of the present invention are in the form of the (−) enantiomer at least 95% free of the corresponding (+) enantiomer.

In one embodiment, the compounds formula (I) present in the pharmaceutical combination of the present invention are in the form of the (−) enantiomer at least 97% free of the corresponding (+) enantiomer.

In one embodiment, the compounds formula (I) present in the pharmaceutical combination of the present invention are in the form of the (−) enantiomer at least 99% free of the corresponding (+) enantiomer.

It will be appreciated by those skilled in the art that the compounds of formula (I) contain at least two chiral centers. The compounds of formula (I) thus exist in the form of two different optical isomers (i.e. (+) or (−) enantiomers or β-L and β-D). All such enantiomers and mixtures thereof including racemic mixtures are included within the scope of the invention. The single optical isomer or enantiomer can be obtained by method well known in the art, such as chiral HPLC, enzymatic resolution and chiral auxiliary. Alternatively, the enantiomers of the compounds of formula (I) can be synthesized by using optically active starting materials.

In one embodiment, the Bcr-Abl tyrosine kinase inhibitor is imatinib mesylate (STI-571).

In one embodiment, the ratio of the compound of formula (I) to the Bcr-Abl tyrosine kinase inhibitor is 1:250 to 250:1, more preferably 1:50 to 50:1, especially 1:20 to 20:1.

In another embodiment, the individual components of such combinations as defined above may be administered either sequentially or simultaneously in separate or combined pharmaceutical formulations.

The combinations referred to above may conveniently be presented for use in the form of a pharmaceutical formulation and thus pharmaceutical formulations comprising a combination as defined above together with a pharmaceutically acceptable carrier therefor comprise a further aspect of the invention.

In one embodiment of the present invention, the compound of formula (I) present in the pharmaceutical combination of the present invention is (β-L-OddC) and the Bcr-Abl tyrosine kinase inhibitor is imatinib mesylate (STI-571). Preferably, the ratio of β-L-OddC to imatinib mesylate (STI-571) is 1:250 to 250:1, more preferably 1:50 to 50:1, especially 1:20 to 20:1.

In one embodiment, the pharmaceutical combination of the present invention is a synergistic combination of therapeutic agents comprising β-L-OddC and imatinib mesylate (STI-571).

In one embodiment, the pharmaceutical combination of the present invention is β-L-OddC and imatinib mesylate (STI-571). Preferably, the ratio of β-L-OddC to imatinib mesylate (STI-571) is 1:250 to 250:1, more preferably 1:50 to 50:1, especially 1:20 to 20:1.

In another embodiment, the present invention provides a combination of the compound of formula (I) and the Bcr-Abl tyrosine kinase inhibitor for treating leukemia selected from the group comprising acute myelogenous leukemia (AML), chronic myelogenous leukemia (CML), acute lymphocytic leukemia (ALL), chronic lymphocytic leukemia (CLL) and hairy cell leukemia (HCL).

In another embodiment, the present invention provides a combination of the compound of formula (I) and the Bcr-Abl tyrosine kinase inhibitor for treating leukemia which is resistant to current drug therapy.

In another embodiment, the present invention provides a combination of β-L-OddC and imatinib mesylate (STI-571) for treating leukemia which is resistant to imatinib mesylate (STI-571).

In another embodiment, the present invention provides a combination of β-L-OddC and imatinib mesylate (STI-571) for treating CML which is resistant to current drug therapy.

In another embodiment, the present invention provides a combination of β-L-OddC and imatinib mesylate (STI-571) for treating CML which is resistant to imatinib mesylate (STI-571).

In one embodiment, the present invention provides a combination as defined above for treating leukemia, wherein there is a further therapeutic agent and the ratio of the compound of formula (I) and the Bcr-Abl tyrosine kinase inhibitor to the further therapeutic agent is preferably 1:250 to 250:1, more referably 1:50 to 50:1, especially 1:20 to 20:1.

In one embodiment, the present invention provides a combination as defined above for treating chronic myelogenous leukemia, wherein there is a further therapeutic agent and the ratio of the compound of formula (I) and the Bcr-Abl tyrosine kinase inhibitor to the further therapeutic agent is preferably 1:250 to 250:1, more preferably 1:50 to 50:1, especially 1:20 to 20:1.

In another embodiment, the present invention provides a combination as defined above for treating refractory/relapsed leukemia, and wherein there is a further therapeutic agent and the ratio of the compound of formula (I) and the Bcr-Abl tyrosine kinase inhibitor to the further therapeutic agent is preferably 1:250 to 250:1, more preferably 1:50 to 50:1, especially 1:20 to 20:1.

In another aspect, the present invention provides a method of treating a patient having leukemia comprising administering to said patient a therapeutically effective amount of a compound of formula (I):

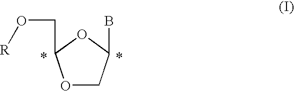

or a pharmaceutically acceptable salt thereof, wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and

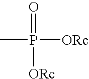

wherein each Rc is independently selected from the group comprising H, C₁₋₆ alkyl, C₂₋₆ alkenyl, C₂₋₆ alkynyl and a hydroxy protecting group;

and a Bcr-Abl tyrosine kinase inhibitor.

In another embodiment, there is provided a method of treating a patient having a leukemia selected from the group of acute myelogenous leukemia (AML), chronic myelogenous leukemia (CML), acute lymphocytic leukemia (ALL), chronic lymphocytic leukemia (CLL) and hairy cell leukemia (HCL) comprising administering to said patient a combination as above.

In another embodiment, the present invention provides a method for treating chronic myelogenous leukemia by administering to the patient a therapeutically effective amount of a compound of formula (I) and a Bcr-Abl tyrosine kinase inhibitor. Preferably, the ratio of the compound of formula (I) to the Bcr-Abl tyrosine kinase inhibitor is 1:250 to 250:1, more preferably 1:50 to 50:1, especially 1:20 to 20:1.

In another embodiment, the present invention provides a method for treating chronic myelogenous leukemia in blastic phase by administering to the patient a therapeutically effective amount of a compound of formula (I) and a Bcr-Abl tyrosine kinase inhibitor. Preferably, the ratio of the compound of formula (I) to the Bcr-Abl tyrosine kinase inhibitor is 1:250 to 250:1, more preferably 1:50 to 50:1, especially 1:20 to 20:1.

In another embodiment, the present invention provides a method for treating refractory/relapsed leukemia by administering to the patient a therapeutically effective amount of a compound of formula (I) and a Bcr-Abl tyrosine kinase inhibitor. Preferably, the ratio of the compound of formula (I) to the Bcr-Abl tyrosine kinase inhibitor is 1:250 to 250:1, more preferably 1:50 to 50:1, especially 1:20 to 20:1.

In another embodiment, the present invention provides a method for treating a patient who has refractory/relapsed leukemia and which has been previously treated with imatinib mesylate (STI-571) by administering to the patient a therapeutically effective amount of a compound of formula (I) and a Bcr-Abl tyrosine kinase inhibitor. Preferably, the ratio of the compound of formula (I) to Bcr-Abl tyrosine kinase inhibitor is preferably 1:250 to 250:1, more preferably 1:50 to 50:1, especially 1:20 to 20:1.

In another embodiment, the present invention provides a method for treating a patient who has refractory/relapsed leukemia and which has been previously treated with imatinib mesylate (STI-571) and is resistant to imatinib mesylate (STI-571) by administering to the patient a therapeutically effective amount of a compound of formula (I) and a Bcr-Abl tyrosine kinase inhibitor. Preferably, the ratio of the compound of formula (I) to the Bcr-Abl tyrosine kinase inhibitor is preferably 1:250 to 250:1, more preferably 1:50 to 50:1, especially 1:20 to 20:1.

In another embodiment, the present invention provides a method for treating a patient who has refractory/relapsed leukemia and which has been previously treated with imatinib mesylate (STI-571) by administering to the patient β-L-OddC and imatinib mesylate (STI-571).

In another embodiment, the present invention provides a method for treating a patient who has refractory/relapsed leukemia and which has been previously treated with imatinib mesylate (STI-571) by administering to the patient β-L-OddC and imatinib mesylate (STI-571) wherein the ratio of β-L-OddC to imatinib mesylate (STI-571) is preferably 1:250 to 250:1, more preferably 1:50 to 50:1, especially 1:20 to 20:1.

In another embodiment, the present invention provides a method for treating a patient with leukemia by administering to the patient a synergistic combination of β-L-OddC and imatinib mesylate (STI-571).

In another embodiment, the present invention provides a method for treating a patient who has refractory/relapsed leukemia and which has been previously treated with imatinib mesylate (STI-571) by administering to the patient a synergistic combination of β-L-OddC and imatinib mesylate (STI-571).

In another embodiment, the present invention provides a method for treating a patient with leukemia by administering to the patient β-L-OddC and imatinib mesylate (STI-571), wherein the ratio of β-L-OddC to imatinib mesylate (STI-571) is preferably 1:250 to 250:1, more preferably 1:50 to 50:1, especially 1:20 to 20:1.

In another embodiment, the present invention provides a method for treating leukemia by administering to the patient a therapeutically effective amount of a compound of formula (I) and a Bcr-Abl tyrosine kinase inhibitor and at least one further therapeutic agent chosen from a nucleoside analogue and/or a chemotherapeutic agent.

There is also provided pharmaceutically acceptable salts of the compounds formula (I) present in the pharmaceutical combinations of the present invention. By the term pharmaceutically acceptable salts of compounds of general formula (I) are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, citric, methanesulphonic, formic, benzoic, malonic, naphthalene-2-sulphonic and benzenesulphonic acids. Other acids such as oxalic, while not in themselves pharmaceutically acceptable, may be useful as intermediates in obtaining the compounds of the invention and their pharmaceutically acceptable acid addition salts.

Salts derived from appropriate bases include alkali metal (e.g. sodium), alkaline earth metal (e.g. magnesium), ammonium and NR₄+ (where R is C₁₋₄ alkyl) salts.

References hereinafter to the pharmaceutical combinations according to the invention includes compounds of the general formula (I) or a pharmaceutically acceptable salt thereof.

Unless otherwise defined, all technical and scientific terms used herein have the same meaning as commonly understood by one of ordinary skill in the art to which this invention belongs. As used in this application, the term “leukemia” represents acute myelogenous leukemia or acute myeloid leukemia (AML), chronic myelogenous leukemia or chronic myeloid leukemia (CML), chronic lymphocytic leukemia (CLL), acute lymphocytic leukemia (ALL), hairy cell leukemia (HCL), myelodysplastic syndromes (MDS) or chronic myelogenous leukemia (CML-BP) in blastic and all subtypes of these leukemias which are defined by morphological, histochemical and immunological techniques that are well known by those of skill in the art.

The term “myelogenous leukemia” represent both acute and chronic myelogenous leukemias (AML, CML) which involve elements of the bone marrow (e.g. white cells, red cells and megakaryocytes) and includes all subtypes of these leukemias which are defined by morphological, histochemical and immunological techniques that are well known by those of skill in the art.

The terms “refractory/relapsed leukemia” represents previously treated patients which were either non responsive to treatment with chemotherapeutic agents or had a response to treatment and then relapsed.

The term “leukemia which is resistant to current therapy” also represents previously treated patients which were either non responsive to treatment with chemotherapeutic agents or had a response to treatment and then relapsed.

The term “patient” is defined as any diseased human.

The term “alkyl” represents an unsubstituted or substituted (by a halogen, nitro, CONH₂, COOH, O—C₁₋₆ alkyl, O—C₂₋₆ alkenyl, O—C₂₋₆ alkynyl, hydroxyl, amino, or COOQ, wherein Q is C₁₋₆ alkyl; C2-6 alkenyl; C₂₋₆ alkynyl) straight chain, branched chain or cyclic hydrocarbon moiety (e.g., methyl, ethyl, n-propyl, isopropyl, butyl, pentyl, hexyl, fluorohexyl, cyclopropyl, cyclobutyl, cyclopentyl, or cyclohexyl). The term alkyl is also meant to include alkyls in which one or more hydrogen atoms is replaced by an halogen, more preferably, the halogen is fluoro (e.g., CF₃— or CF₃CH₂—).

The terms “alkenyl” and “alkynyl” represent an alkyl containing at least one unsaturated group (e.g., vinyl, 1-propenyl, allyl, 1-methylpropenyl, 2-butenyl, 2-butenyl, ethynyl, 1-propynyl, or 2-propynyl).

The term “aryl” represents an aromatic radical (e.g., phenyl and naphthyl).

The term “hydroxy protecting group” is well known in the field of organic chemistry. Such protecting groups may be found in T. Greene, Protective Groups In Organic Synthesis, (John Wiley & Sons, 1981). Example of hydroxy protecting groups include but are not limited to acetyl-2-thioethyl ester, pivaloyloxymethyl ester and isopropyloxycarbonyloxymethyl ester.

In one embodiment, the first compound of formula (I) is administered to the patient at a dose between about 1 mg/m² and about 8 mg/m²; and the Bcr-Abl tyrosine kinase inhibitor is administered to the patient at a dose between about 0.1 gm/m² and about 30 gm/m².

In one embodiment, the first compound of formula (I) is administered to the patient at a dose between about 1 mg/m² and about 8 mg/m²; and the Bcr-Abl tyrosine kinase inhibitor is administered to the patient at a dose between about 0.1 gm/m² and about 6 gm/m².

In one embodiment, β-L-OddC is administered to the patient at a dose between about 1 mg/m² and about 8 mg/m²; and imatinib mesylate (STI-571) is administered to the patient at a dose between about 0.1 gm/m² and about 30 gm/m².

In one embodiment, β-L-OddC is administered to the patient at a dose between about 1 mg/m² and about 8 mg/m²; and imatinib mesylate (STI-571) is administered to the patient at a dose between about 0.1 gm/m² and about 6 gm/m².

In another embodiment, β-L-OddC is administered at 6mg/m² over 30 minutes per day on days 1 to 5 and imatinib mesylate (STI-571) is administered at 1 gm/m² over 2 hours daily on days 1 to 5.

In another embodiment, β-L-OddC is administered at 5mg/m² over 30 minutes per day on days 1 to 5 and imatinib mesylate (STI-571) is administered at 21 gm/m² over 2 hours daily on days 1 to 3.

It will be appreciated that the amount of pharmaceutical combination according to the invention required for use in treatment will vary not only with the particular compound selected but also with the route of administration, the nature of the condition for which treatment is required and the age and condition of the patient and will be ultimately at the discretion of the attendant physician. In general however, a suitable dose will be in a range of from about 0.1 to about 750 mg/kg of body weight per day, preferable in the range of 0.5 to 500 mg/kg/day, most preferably in the range of 1 to 300 mg/kg/day.

The desired dose may conveniently be presented in a single dose or as divided dose administered at appropriate intervals, for example as two, three, four or more doses per day.

The pharmaceutical combination according to the present invention is conveniently administered in unit dosage form.

Ideally the active ingredient should be administered to achieve peak plasma concentrations of the active compound of from about 1 to about 75 μM, preferably about 2 to 50 μM, most preferably about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

The combinations referred to above may conveniently be presented for use in the form of a pharmaceutical formulation and thus pharmaceutical formulations comprising a combination as defined above together with a pharmaceutically acceptable carrier therefor comprise a further aspect of the invention.

The individual components of such combinations may be administered either sequentially or simultaneously in separate or combined pharmaceutical formulations.

When the compound (I) or a pharmaceutically acceptable salts thereof is used in combination with a second therapeutic agent the dose of each compound may be either the same as or differ from that when the compound is used alone. Appropriate doses will be readily appreciated by those skilled in the art.

For advantageous effects of the combination of the compounds of formula (I) and the Bcr-Abl tyrosine kinase inhibitor and the additional therapeutic agents, they may be administered over a wide ratio. In one embodiment, the ratio of the compounds of formula (I) to the additional therapeutic agents in the present invention is between 1:250 to 250:1.

In a further embodiment, one may use from about 1:1 to about 1:15 of compounds of the invention:second therapeutic agent. In a further embodiment, one may use from about 1:1 to about 1:10 of compounds of the invention:second therapeutic agent. In a further embodiment, one may use from about 1:1 to about 1:5 of compounds of the invention:second therapeutic agent. In a further embodiment, one may use from about 1:1 to about 1:3 of compounds of the invention:second therapeutic agent. If a further therapeutic agent is added, ratios will be adjusted accordingly.

While it is possible that, for use in therapy, a compound of the invention may be administered as the raw chemical it is preferable to present the active ingredient as a pharmaceutical formulation. The invention thus further provides a pharmaceutical formulation comprising a compound of formula (I) or a pharmaceutically acceptable derivative thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

Pharmaceutical formulations include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical formulation suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical combination according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing an/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

The pharmaceutical combination according to the invention may also be formulated for direct administration to the Central Nervous System by intravenous administration. In addition, administration to the heart may be achieved.

For topical administration to the epidermis, the pharmaceutical combination according to the invention may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active ingredients in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid are most preferably presented as unit dose suppositories. Suitable carriers include cocoa butter and other materials commonly used in the art, and the suppositories may be conveniently formed by admixture of the active compounds with the softened or melted carrier(s) followed by chilling and shaping in moulds.

Formulations suitable for vaginal administration may be presented as pessaries, tampons, creams, gels, pastes, foams or sprays containing in addition to the active ingredient such carriers as are known in the art to be appropriate.

For intra-nasal administration the pharmaceutical combination according to the invention may be used as a liquid spray or dispersible powder or in the form of drops. Drops may be formulated with an aqueous or non-aqueous base also comprising one more dispersing agents, solubilising agents or suspending agents. Liquid sprays are conveniently delivered from pressurized packs.

For administration by inhalation the pharmaceutical combination according to the present invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the pharmaceutical combination according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

The entire disclosure of all applications, patents and publications, cited above and below, is hereby incorporated by reference.

The following examples are provided to illustrate various embodiments of the present invention and shall not be considered as limiting in scope.

Compounds

The compounds of formula (I), including but not limited to Troxatyl™ (β-L OddC), was synthesized at Shire BioChem Inc. as previously described in PCT publication numbers WO96/07413A1, WO97/21706 and WO00/47759, all of which are hereby incorporated by reference. Imatinib mesylate (STI-571) was obtained from ovartis.

Cell Lines

Two human CML, Ph+, p210 Bcr-Abl expressing cell lines were used, namely, KBM-5 and KBM-7. KBM-5 represents cells derived from a patient in the blastic phase of CML and is remarkable for the absence of normal c-ABL. KBM-7 has been identified to be a human near-haploid cell line. These two cell lines were previously described in the references below, now incorporated by reference:

Beran M., Pisa p., O'Brien S., Kurzrock R., Siciliano M., Cork A., Andersson B S., Kohli V., Kantarjian H., Biological Properties and growth in SCID mice of a new myelogenous leukemia cell line (KBM-5) derived from chronic myelogenous leukemia cells in the blastic phase. Cancer Research, 53 (15): 3603-3610, 1993.

Kotecki M., Reddy P S., Cochran B H., Isolation and characterization of a near-haploid human cell line, Exp. Cell. Res., 252(2): 273-280, 1999

Andersson B S., Collins V P., Kurzrock R., Larkin D W., Childs C., Ost A., Cork A., Trujillo J M., Freireich E J., Siciliano M J., Leukemia, 9(12): 2100-2108, 1995.

The KBM-5 and KBM-7 cells differ in their inherent sensitivity to STI-571 and in their response to STI-571 exposure. The cells were cultured in Iscove's modified Dulbecco's medium supplemented with 10% fetal calf serum (Invitrogen Corp., Carlsbad, Calif.) at 37° C. in atmosphere of 5% CO₂ in air. These cells also differ in their response to STI-571 exposure: G0/G1 cell cycle arrest in KBM5 vs. apoptosis in KBM7. The effective dose of STI-571 which kills 50% of KBM-5 cells was 0.6 μM and for KBM-7 it was 0.3 μM.

Generation of STI-571-resistant KBM5 and KBM7 Ph+ Cell Lines

STI-571 resistant cell lines were developed by culturing the cells with increasing concentrations of STI-571, as described in detail below. Cells maintained in liquid cultures were exposed to increasing concentrations of STI-571, starting with a concentration of 0.05 μM, and increasing gradually at a rate of 0.1 μM. When the survival of the cells grown in a given STI-571 concentration reached 80%, a proportion of cells were frozen while the remaining cells were grown at a next higher drug level. In this way, subpopulations of cells with different degree of resistance were generated (e.g, KBM5-STIR^{0.75} indicating KMB5 cells resistant to STI-571 at the dose of 0.075 μM). The resistance was defined as the ability of cells to survive (at least 80% survival) and proliferate indefinitely in continuous presence of a given concentration of STI-571. The resistant cells emerged earlier in KBM5 than in KBM7 cells and this reflected the lower inherent sensitivity of these cells. Thus, KBM5 cells were able to survive in 1.0 μM of STI-571 4 months after the initiation of the experiments, whereas a similar level or resistance was reached only after 10 months in KBM7 cells. KBM5-STIR^{R1.0} and KBM7-STI^{R1.0}, the sublines with the highest level of resistance, showed an IC₅₀ about twenty times higher than the value calculated in the corresponding parental cell line. Interestingly, increasing the concentration of STI-571 to even higher levels revealed that KBM5-STI^{R1.0} stil proliferated at concentrations up to 10 μM, whereas the proliferation of KBM7-STI was almost completely abolished at 7.5 μM. Therefore, the effective dose of STI-571 which killed 50% of KBM5-STI^{R1.0} cells was 10 μM and for KBM7-STI^{R1.0}, the effective dose was 3 μM.

Growth Inhibition (MTS) Assay

In vitro growth inhibition effect of adaphostin on leukemic cells was determined by measuring MTS (CellTiter 96®Aqueous One Solution Reagent, Promega Corporation, WI) dye absorbance by living cells. Briefly, cells were seeded in triplicate in 96-well microtiter plates (Falcon, USA) at a concentration of 4×10⁵ cells/ml. After exposure to the drug(s) for 72 h, 20 μl of MTS solution were added to each well, the plates were incubated for additional 4 h at 37° C., and absorbance at 490 nm was measured. In combination experiments the dose of Troxatyl™ varied while the dose of STI-571 stayed fixed. The dose of STI-571 used in combination experiments was just enough to kill 10-20% of respective cells.

Caspase-3/7 Assay

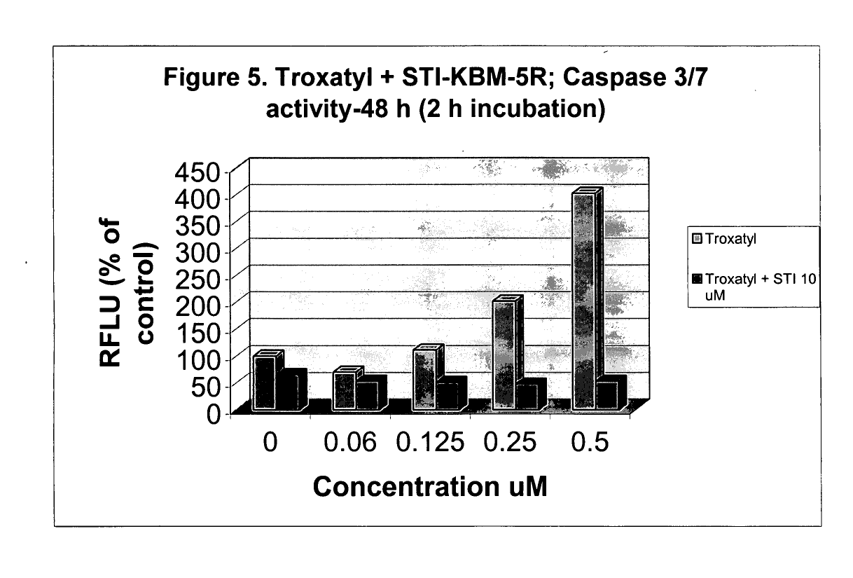

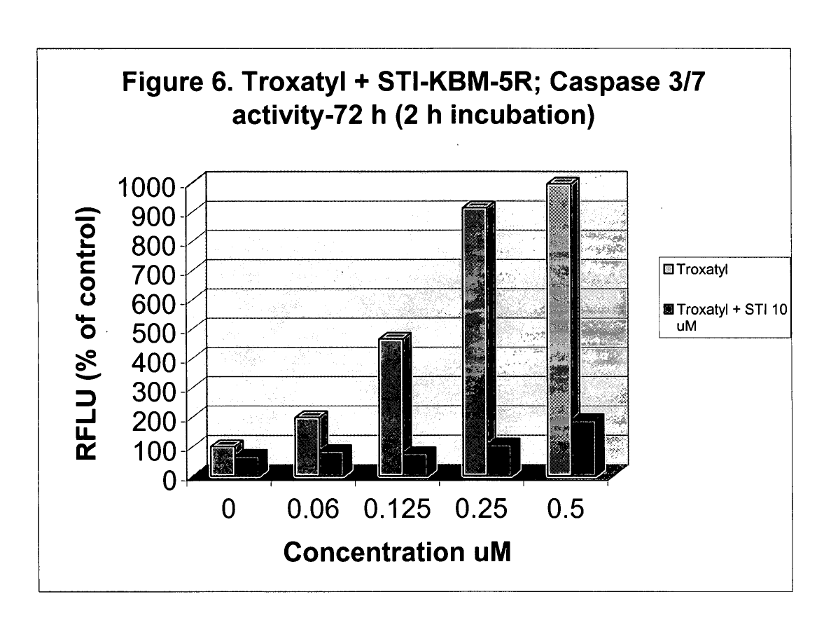

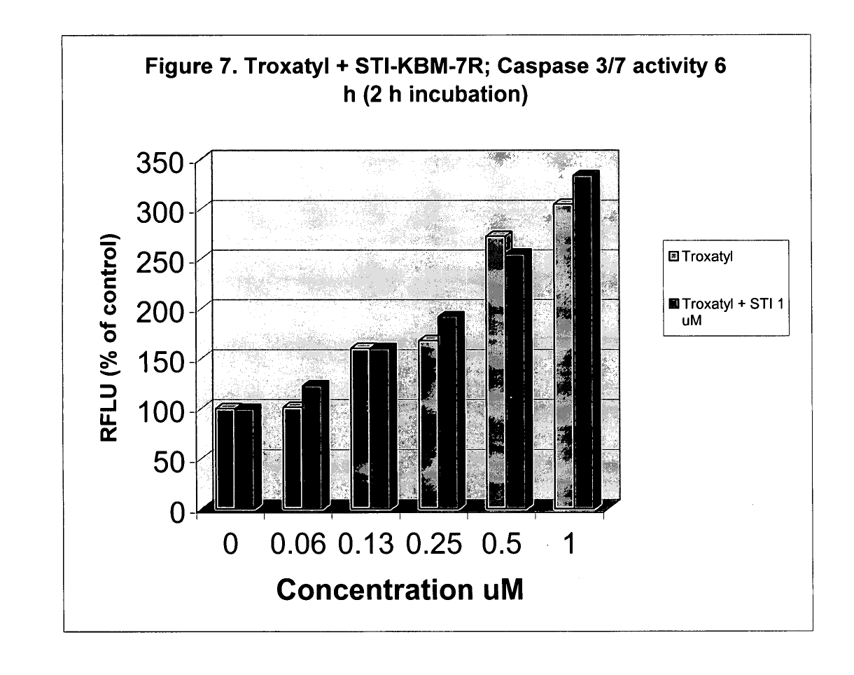

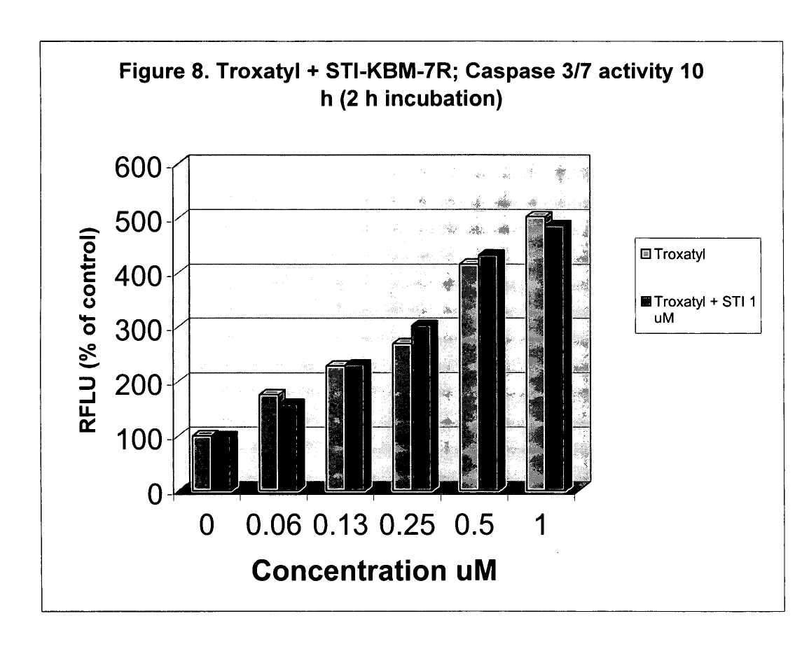

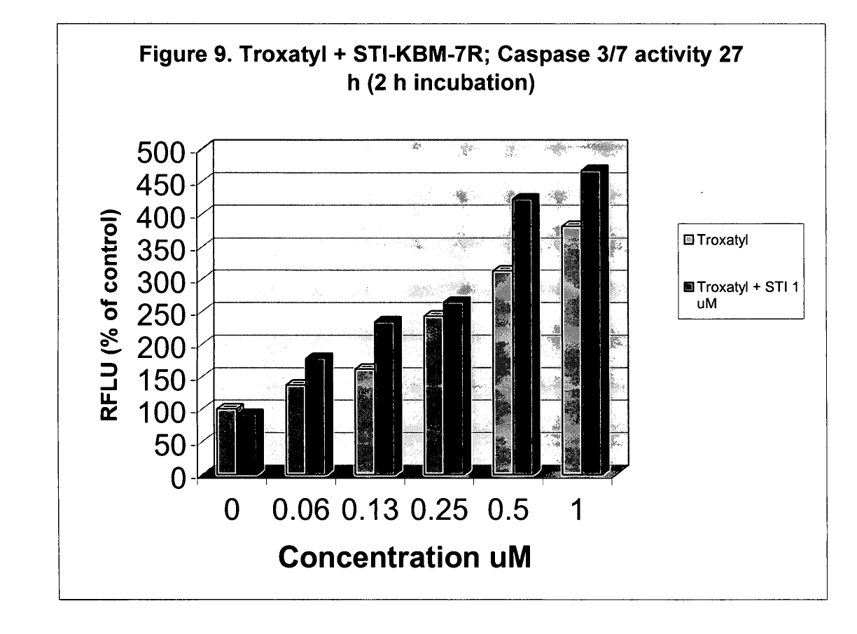

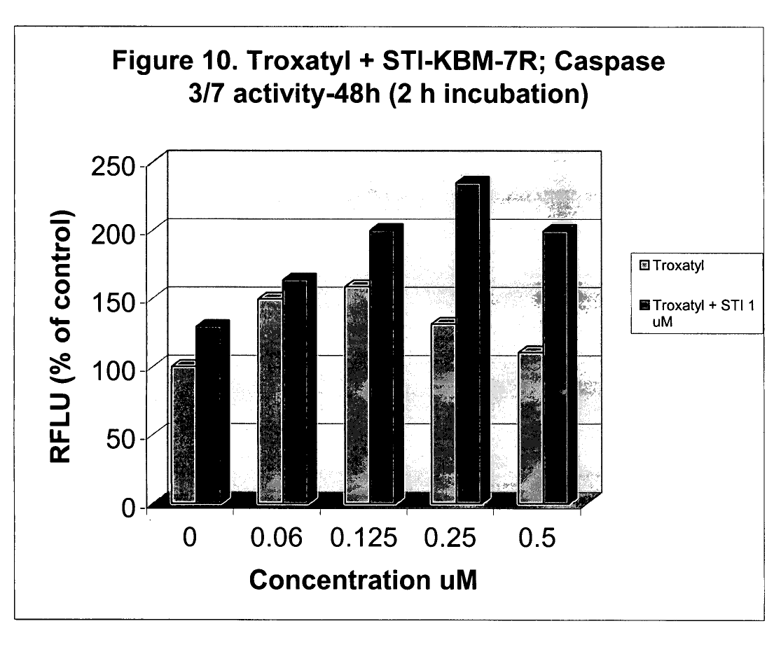

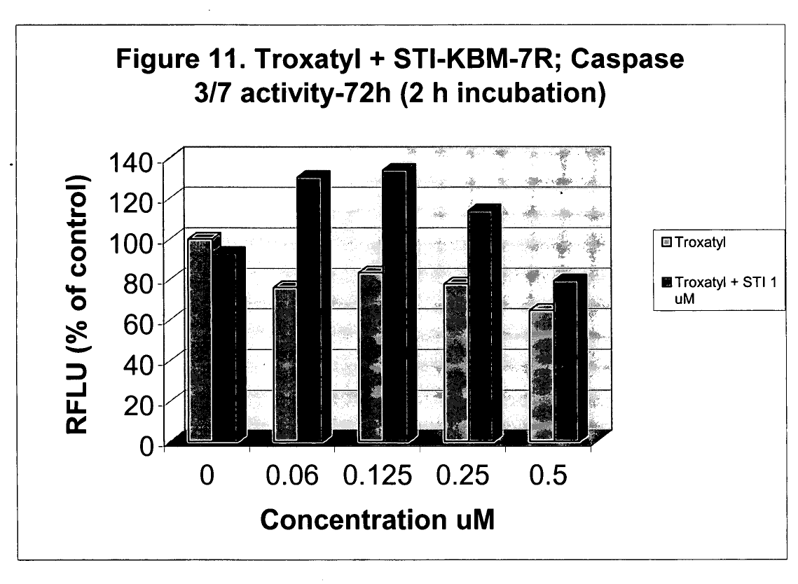

Caspase activity was measured with the Apo-One™ Homogeneous Caspase 3/7-assay kit (Promega Corporation, Madison, Wis.). This assay uses fluorogenic substrate rhodamine 110, bis- (N-CBZ-L-aspartyl-glutaml-L-valyl-L-aspartic acid amide) (Z-DEVD-R110). Caspase activity was assayed by detection of free rhodamine 110 group upon sequential cleavage and removal of the DEVD peptides by caspase-3/7. Cleavage of the fluorogenic caspase 3/7 substrate Z-DEVD-R110 was performed according to manufacturer's instructions, using a Fluorostar plate reader and excitation and emission wavelengths of 499 nm and 521 nm, respectively. Briefly, cells were seeded at a density of 1.5×10⁶/ml and incubated in the presence or absence of drug(s) for indicated time. Homogeneous caspase-3/7 reagent was added to an aliquot of cell culture in a 96-well plate and reaction mixture was incubated for 2 h at room temperature before measurement of fluorescence. The results of these experiments are seen in FIGS. 5 to 11.

In Vivo Studies

Three to five week-old ICR SCID female mice weighting 20-25 g were obtained from Taconic farms. They were acclimatized for a week prior to the experiment. The animals were maintained on a standard animals feed and drinking water ad libitum. Mice were housed in an air-conditioned room at the temperature of 22±1° C. and 50-70% humidity with a 12/12 h-light/dark cycle throughout the experiment.

Mice were irradiated (1×250 centigray; cGy) and injected i.p. with 2×10⁷ or 2,4×10⁷ KBM-5 or KBM-5R tumor cells, respectively. Treatment with Troxatyl™ was started 20 days or after 25 days with STI-571 after KBM-5 (chronic myeloid leukaemia cells) or KBM-5R (chronic myeloid leukaemia cells resistant to STI-571) tumor cell injections, once the mice had developed visible tumors at the site of inoculation. In primary experiments (with KBM-5 cells) or secondary experiments (with KBM-5R cells), tumor-bearing animals were randomised (8-10 per group and treated by Troxatyl™ i.p. at 5, 10, 20, or 25 mg/kg once a day for 5 consecutive days (days 20-24). Control (untreated) mice received saline. In third experiments (with KBM-5 cells), tumor bearing animals (6 per group) were treated by one of the following schemes:

a) control (saline i.p.);

b) Troxatyl™ (10 mg/kg per day i.p.);

c) Troxatyl™ (25 mg/kg per day i.p.);

d) STI-571 (50 mg/kg per day i.p.);

e) Troxatyl™+STI-571 (10 mg/kg+50 mg/kg);

f) Troxatyl™+STI-571 (25 mg/kg+50 mg/kg).

In this study, treatment was given once a day for 5 consecutive days (days (20-24) for Troxatyl™ or twice a day for 10 consecutive days (days 25-34) for STI-571. For the survival analysis, the death endpoint was determined either by spontaneous death of the animals or by elective killing (with CO gas) of the animal because of signs pain or suffering according to established criteria. Results are expressed as percent of mean survival time of treated animals over mean survival time of the control group (treated vs. control, T/C%) and increased lifespan (mean survival time of treated animals minus that of control animals over the mean survival time of the control group; increased life span, ILS, %). By NCI criteria, T/C exceeding 125% and ILS exceeding 25% indicate that the drug has significant antitumor activity (Plowman et al. 1995). Almost of the spontaneous death animals and all of survival animals after the survival studies were killed well to perform analysis for human DQα-gen. Animals in complete remission, free of detectable tumor (negative for human DQα-gen) were considered cured.

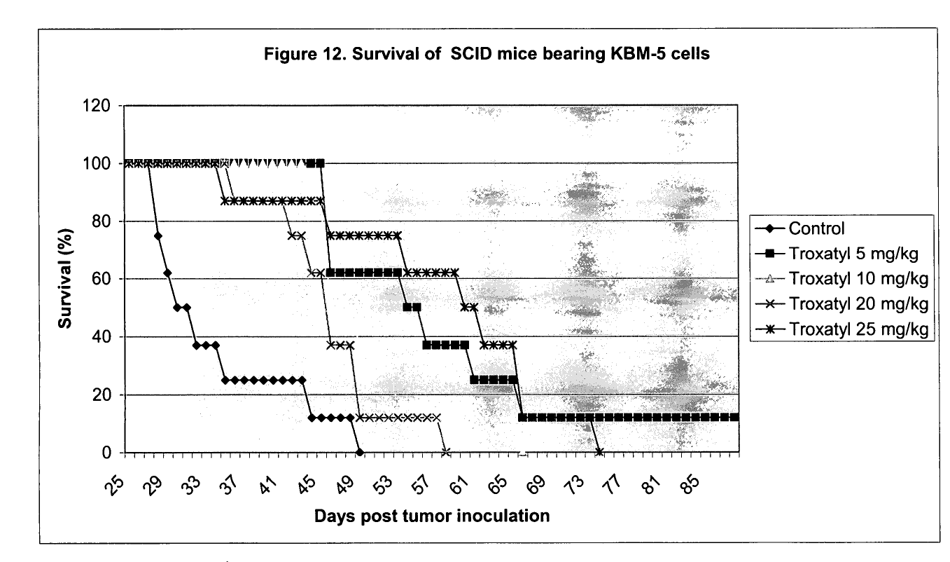

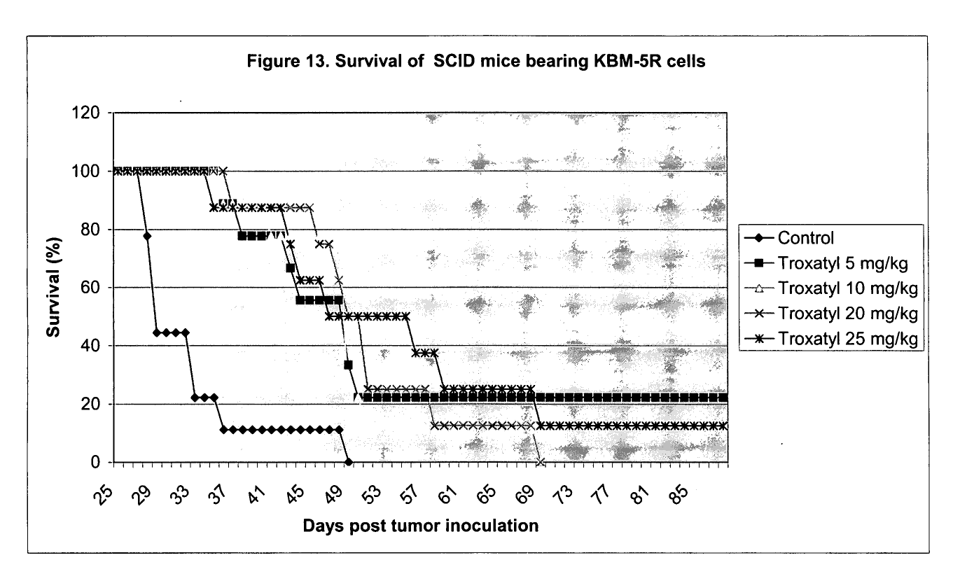

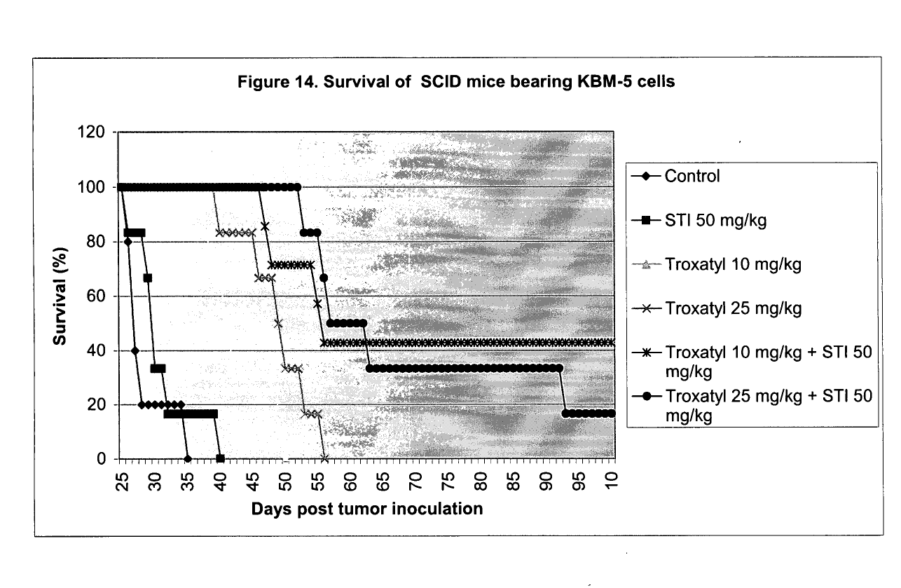

Table 1 and FIGS. 12 to 14 show the results of the in vivo studies. The results of the study show that the combination of Troxatyl™ with STI-571 gives a synergistic result in the KBM-5 cell line. (LTS means long term survivors)

TABLE 1
Comparative in vivo antitumor activity of Troxatyl
with or without STI571 treatment in mice bearing KBM-5 or

| | | | | | Rang | Median | | | |
|---|---|---|---|---|---|---|---|---|---|
| | | Mice | Dose | | survival | survival | | | |
| | Cell | per | mg/kg | | time | time | | | |
| Groups | lines | group | (ip) | Schedule | (days) | (days) | ILS % | T/C % | LTS |
| Control | KBM-5 | 8 | Saline | qd × 5 | 28-49 | 34.375 | — | — | |
| Troxatyl ™ | | 8 | 5 | | 46-66 | 54 | 57.09 | 157.09 | 1 |
| | | 8 | 10 | | 44-66 | 50 | 45.45 | 145.45 | |
| | | 8 | 20 | | 36-58 | 46 | 33.82 | 133.82 | |
| | | 8 | 25 | | 35-74 | 58 | 68.73 | 168.73 | |
| Control | KBM- | 9 | Saline | qd × 5 | 28-49 | 32.6 | — | — | |
| Troxatyl ™ | 5R | 9 | 5 | | 36-50 | 37.142 | 13.93 | 113.93 | 2 |
| | | 9 | 10 | | 36-69 | 49.11 | 50.64 | 150.64 | |
| | | 8 | 20 | | 37-69 | 51.13 | 56.84 | 156.84 | |
| | | 8 | 25 | | 35-69 | 50.42 | 54.66 | 154.66 | 1 |
| Control | KBM-5 | 5 | Saline | qd × 5 | 26-35 | 28.6 | — | — | |
| Troxatyl ™ | | 6 | 10 | | 38-50 | 43.16 | 50.90 | 150.9 | |
| | | 6 | 25 | | 40-56 | 49 | 71.33 | 171.33 | |
| STI-571 | | 6 | 50 | bid × 10 | 26-40 | 31.16 | 8.95 | 108.95 | |
| Troxatyl ™ + STI-571 | | 7 | 10 + 50 | qd × 5 + bid × 10 | 47-56 | 51.5 | 80.06 | 180.06 | 3* |
| | | 6 | 25 + 50 | | 53-93 | 64.4 | 125.17 | 225.17 | 1* |

KBM-5R chronic myeloid leukemia cells

Female ICR SCID 3-5 weeks old mice were injected ip with 2.4×10⁷ KBM-5 or KBM-5R (STI-571 resistant) tumor cells on Day 0. Treatment with Troxatyl™ (daily for 5 days) started on Day 20 and treatment with STI-571 (twice a day for 10 days) started on Day 25. LTS were electively sacrificed on Day 95 in single agent experiments or on Day 100 in combination treatment experiment.

PCR analysis for human HLA-DQα gen was performed on spleen, liver, bone marrow or tumor tissue from LTS and most other mice. All examined mice that were not LTS had leukemia. Results of PCR showed that LTS in single agent experiments had no leukemia indicating most likely failure of leukemia engraftment into mice. LTS in combination therapy experiment (indicated by *) had positive PCR in bone marrow indicating presence of minimal disease.

What is claimed is

1. A pharmaceutical combination comprising at least one active compound of formula (I): 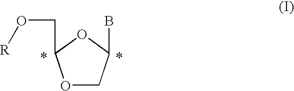
or a pharmaceutically acceptable salt thereof,
wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and 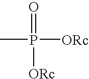
wherein each Rc is independently selected from the group comprising H, C₁₋₆ alkyl, C₂₋₆ alkenyl, C₂₋₆ alkynyl and a hydroxy protecting group;
and a Bcr-Abl tyrosine kinase inhibitor.

2. The pharmaceutical combination according to claim 1, wherein the Bcr-Abl tyrosine kinase inhibitor is imatinib mesylate (STI-571).

3. The pharmaceutical combination according to claim 2, wherein R is H.

4. The pharmaceutical combination according to claim 2, wherein B is cytosine.

5. The pharmaceutical combination according to claim 2, wherein R is H and B is cytosine.

6. The pharmaceutical combination according to claim 2, wherein B is 5-fluorocytosine.

7. The pharmaceutical combination according to claim 2, wherein the compound of formula I is (−)-β-L-Dioxolane-Cytidine (β-L-OddC).

8. The pharmaceutical combination according to claim 2, wherein the compound of formula I is (−)-β-Dioxolane-5-fluoro-Cytidine (5-FddC).

9. The pharmaceutical combination according to claim 2, wherein the compound of formula I is substantially in the form of the (−) enantiomer.

10. The pharmaceutical combination according to claim 2, wherein said compound of formula (I) is at least 97% free of the corresponding (+) enantiomer.

11. The pharmaceutical combination according to claim 2 wherein the compound of formula (I) is β-L-OddC and the Bcr-Abl tyrosine kinase inhibitor is imatinib mesylate (STI-571).

12. A pharmaceutical combination according to claim 2 wherein the compound of formula (I) and imatinib mesylate (STI-571) are present in a ratio between about 1:50 to about 50:1.

13. A pharmaceutical combination according to claim 2 wherein the compound of formula (I) and imatinib mesylate (STI-571) are present in a ratio between about 1:20 to about 20:1.

14. A pharmaceutical combination comprising at least one active compound of formula (I): 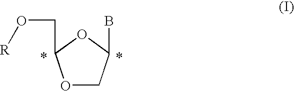
or a pharmaceutically acceptable salt thereof,
wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and 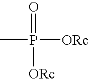
wherein each Rc is independently selected from the group comprising H, C₁₋₆ alkyl, C₂₋₆ alkenyl, C₂₋₆ alkynyl and a hydroxy protecting group;
and a Bcr-Abl tyrosine kinase inhibitor and the compound of formula (I) and the Bcr-Abl tyrosine kinase inhibitor are present in a synergistic ratio.

15. A method of treating a patient having leukemia comprising administering to said patient a therapeutically effective amount of a compound of formula I: 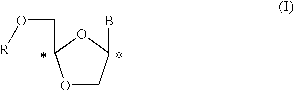
or a pharmaceutically acceptable salt thereof,
wherein B is cytosine or 5-fluorocytosine and R is selected from the group comprising H, monophosphate, diphosphate, triphosphate, carbonyl substituted with a C₁₋₆ alkyl, C₂₋₆ alkenyl, C₂₋₆ alkynyl, C₆₋₁₀ aryl and 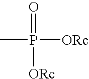
wherein each Rc is independently selected from the group comprising H, C₁₋₆ alkyl, C₂₋₆ alkenyl, C₂₋₆ alkynyl and a hydroxy protecting group;
and Bcr-Abl tyrosine kinase inhibitor.

16. A method of treating a patient having leukemia according to claim 15 and wherein the ratio of the compound of formula (I) and the Bcr-Abl tyrosine kinase inhibitor is 1:250 to 250:1.

17. The method according to claim 15, wherein the step of administering comprises administering to a patient with acute myelogenous leukemia and chronic myelogenous leukemia.

18. The method according to claim 15, wherein the step of administering comprises administering to a patient with chronic myelogenous leukemia in blastic phase.

19. The method according to claim 15, wherein the step of administering comprises administering to a patient with refractory/relapsed leukemia.

20. The method according to claim 15, wherein the step of administering comprises administering to a patient with refractory/relapsed leukemia and which has been previously treated with imatinib mesylate (STI-571).

21. The method according to claim 15, wherein the step of administering comprises administering to a patient with refractory/relapsed leukemia and which has been previously treated with imatinib mesylate (STI-571) and is resistant to imatinib mesylate (STI-571).

22. The method according to claim 15, wherein the step of administering comprises administering to a patient with refractory/relapsed leukemia and which has been previously treated with imatinib mesylate (STI-571) wherein the compound of formula (I) is β-L-OddC and the Bcr-Abl tyrosine kinase inhibitor is imatinib mesylate (STI-571).

23. The method according to claim 15, wherein the step of administering comprises administering to a patient with refractory/relapsed leukemia and which has been previously treated with imatinib mesylate (STI-571) and wherein the compound of formula (I) is β-L-OddC and the Bcr-Abl tyrosine kinase inhibitor is imatinib mesylate (STI-571) and said combination is a synergistic combination.

24. A method of treating a patient having cancer, other than leukemia, comprising administering to said patient a therapeutically effective amount of a compound of formula I: 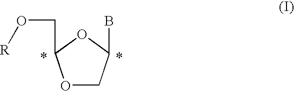
or a pharmaceutically acceptable salt thereof,
wherein B is cytosine or 5-fluorocytosine and R is selected from the group comprising H, monophosphate, diphosphate, triphosphate, carbonyl substituted with a C₁₋₆ alkyl, C₁₋₆ alkenyl, C₂₋₆ alkynyl, C₆₋₁₀ aryl and 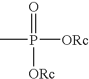
wherein each Rc is independently selected from the group comprising H, C₁₋₆ alkyl, C₂₋₆ alkenyl, C₂₋₆ alkynyl and a hydroxy protecting group;
and a Bcr-Abl tyrosine kinase inhibitor;
and at least one further therapeutic agent chosen from a nucleoside analogue and/or a chemotherapeutic agent.

25. A pharmaceutical composition comprising a pharmaceutical combination according to claim 1 and at least one pharmaceutically acceptable carrier or excipient.
